(19) United States
(12) Patent Application Publication
(10) Pub. No.: US 2014/0330131 A1
(43) Pub. Date: Nov. 6, 2014

(54) METHOD OF AND A SYSTEM FOR CHARACTERISING A MATERIAL

(71) Applicants: Alexandre Francois, North Adelaide (AU); Tanya Mary Monro, Urrbrae (AU); Kristopher Rowland, Glenalta (AU)

(72) Inventors: Alexandre Francois, North Adelaide (AU); Tanya Mary Monro, Urrbrae (AU); Kristopher Rowland, Glenalta (AU)

(73) Assignee: ADELAIDE RESEARCH & INNOVATION PTY LTD., Adelaide (AU)

(21) Appl. No.: 14/117,135

(22) PCT Filed: May 14, 2012
(86) PCT No.: PCT/AU12/00521

§ 371 Date: Jun. 23, 2014

(30) Foreign Application Priority Data
May 13, 2011 (AU) 2011901833

(51) Int. Cl. G01J 1/04; A61B 1/07; A61B 5/00

(52) U.S. Cl. CPC G01J 1/0403; G01J 1/0425; A61B 5/0084; A61B 1/07
USPC 600/478; 250/227.11

(57) ABSTRACT

A system for characterising a material is provided. The system includes an optical sensor including an optical waveguide, the optical waveguide having first and second ends and being characterised by having a numerical aperture greater than or equal to 0.2, and a microresonator including an optically active material, the microresonator being positioned in an optical near field of an end face of the first end of the optical waveguide such that the optically active material is excitable by light. The system further includes a light source for exciting the optically active material of the microresonator so as to generate whispering gallery modes (WGMs) in the microresonator and a light collector for collecting an intensity of light that is associated with the WGMs excited in the microresonator.

### FIELD OF THE INVENTION

The present invention relates to a method of and a system for characterizing a material.

### BACKGROUND OF THE INVENTION

Microresonators, such as microspheres, can be used for sensing purposes, such as temperature sensing. However, using microresonators for sensing applications in the liquid phase typically requires a microfluidic flow cell to flow samples around the microsphere and consequently in-vivo sensing using microresonators is difficult to implement.

As such, there is a need for technological advancement.

### SUMMARY OF THE INVENTION

In accordance with a first aspect of the present invention, there is provided a system for characterising a material, the system comprising:
an optical sensor comprising an optical waveguide, the optical waveguide having first and second ends and being characterised by having a numerical aperture greater than or equal to 0.2, the optical sensor further comprising a microresonator, the microresonator comprising an optically active material and being positioned in an optical near field of an end face of the first end of the optical waveguide such that the optically active material is excitable by light;
a light source for exciting the optically active material of the microresonator so as to generate whispering gallery modes (WGMs) in the microresonator; and
a light collector for collecting an intensity of light that is associated with the WGMs excited in the microresonator.

The system typically is arranged for in-vivo and/or in-vitro biosensing, such as by coating the microresonator with a material that is arranged to interact with a particular biomolecule.

The microresonator may be in contact with the end face of the first end of the optical waveguide, or the microresonator may be spaced from the end face of the first end of the optical waveguide by a distance of 10 μm or less.

It will be appreciated that the end face of the first end of the optical waveguide may have any appropriate orientation. For example, a plane of the end face may be substantially perpendicular with respect to a length of the optical waveguide, or the plane of the end face may be oblique with respect to the length of the optical waveguide. It will also be appreciated that the first end of the optical waveguide may be tapered.

The waveguide may be characterised by having a numerical aperture greater than or equal to any one of the group comprising 0.2, 0.5, 0.75, 1.0, 1.25, 1.5 and 1.75, or within the range of any one of the group comprising 0.2-3.0 and 0.2-1.75.

Throughout the specification, the term “numerical aperture” is used to quantify a characteristic of a waveguide, a numerical aperture having a standard definition of:

NA=\sqrt{n₁²−n₂²} Equation 1

where NA is the numerical aperture, n₁ is the refractive index of a core of the waveguide and n₂ is the refractive index of a cladding of the waveguide that is immediately adjacent the core. For a microstructure optical fibre (MOF) n₁ is the glass index and n₂ is approximately equal to 1 (air).

The numerical aperture is also related to θₘₐₓ, a maximum angle an external light ray can make with an end of the waveguide and still be guided, by:

NA=n₀ sin(θₘₐₓ)

where n₀ is the refractive index of an environment light exiting the waveguide enters. If the end of the waveguide is in air, n₀ would be approximately equal to 1. If the end of the waveguide is positioned in an aqueous environment, n₀ may be approximately equal to 1.33. It will be appreciated that the end of the waveguide may be positioned in a medium of arbitrary index.

Any incoming ray with an angle of incidence greater than θₘₐₓ will not be totally internally reflected within the waveguide and hence not guided. This maximum acceptance angle defines the ‘acceptance cone’ of an optical fibre. Larger capture efficiencies require larger values of NA (larger acceptance cone). It will be appreciated that Equation 1 is not strictly valid as a measure of the acceptance/emission cone for small core MOFs due to diffraction effects on these small scales but that the numerical aperture can still be a useful guide to the behaviour of small core MOFs.

Such a system provides the significant advantage of providing a sensor that can function as, for example, a dip sensor, wherein the waveguide is used for both directing light to the microresonator so as to excite WGMs in the microresonator and for collecting an intensity of light that comprises at least a portion of the excited WGMs.

Further, optically coupling the microresonator to the waveguide having a numerical aperture greater than or equal to 0.2 provides the significant advantage of increasing the excitation and collection efficiency of a WGM signal generated by the microresonator compared to a typical sensor such as a microresonator embedded into a microfluidic flow cell.

The optically active material is typically a material which absorbs light at a certain wavelength and re-emits light at a higher wavelength. For example, the optically active material may comprise an organic dye, a quantum dot, or a rare earth ion. In one specific example, the optically active material is a fluorescent dye, such as Nile Red. In another specific example, the optically active material is a rare earth doped material, such as a rare earth doped glass or a rear earth doped polymer.

In one embodiment, the optical waveguide is an optical fibre, however it will be appreciated that the waveguide could be any appropriate waveguide such as a planar waveguide.

The waveguide may be an optical fibre comprising a core having a diameter equal to or less than 100 μm, such as less than 50 μm, 20 μm, 10 μm or 5 μm. In one specific example, the core of the optical fibre has a diameter of approximately 1.5 μm. The optical fibre may be a microstructured optical fibre (MOF).

The MOF may comprises a glass having a refractive index that is equal to or greater than any one of the group comprising 1.4, 1.55, 2 and 2.5.

The MOF may comprise one or more holes that extend along an axis of the optical fibre. The MOF may comprise a solid core, or the MOF may comprise a hollow core.

For embodiments wherein the MOF comprises one or more holes that extend along an axis of the optical fibre, the microresonator may be associated with at least one hole of the MOF. In one example, the microresonator is anchored to one of the holes of the MOF.

In one example, the waveguide is a multi-core optical fibre and the system is arranged such that a first core is used in the excitation of WGMs in the microresonator and a further core is used in collecting an intensity of light that is associated with the WGMs excited in the microresonator.

The microresonator may be a microsphere. In one embodiment, the microresonator comprises a polymer. In a particular example, the microresonator comprises polystyrene. In another embodiment, the microresonator comprises silica.

In one embodiment, the microresonator has a diameter in the range of 1 μm-50 μm. The microresonator may have a diameter in the range of 5 μm-15 μm or in the range of 9 μm-11 μm. In one example, the microresonator has a diameter of 10 μm.

In one embodiment, the microresonator is arranged so as to be operable in the lasing regime.

Having an optical sensor comprising a microresonator arranged so as to be operable in the lasing regime provides the significant advantage of increasing a sensitivity at which the microresonator reacts to changes in its environment.

The microresonator may be coupled to a resonator, such as a further microresonator.

In one embodiment, the sensor comprises a plurality of microresonators positioned in an optical near field of an end face of the first end of the waveguide, at least two microresonators being arranged so as to interact with different material particles. In one example, at least some microresonators are surface functionalised so as to enable the at least some microresonators to interact with the same and/or different material particles. At least some microresonators may comprise the same optically active material, such as the same fluorescent dye, such that the least some microresonators emit within the same wavelength range. In an alternative embodiment, a first group of microresonators comprise an optically active material that emits within a first frequency range, such as a first fluorescent dye, and a second group of microresonators comprise an optically active material that emits within a second frequency range, such as a second fluorescent dye, thereby allowing the first and the second groups of microresonators to be excited separately.

In one embodiment, the waveguide comprises a wagon wheel or small core microstructured optical fibre architecture.

In one embodiment, the waveguide is a hollow core fibre having a core diameter that is of the same order as a diameter of the microresonator, the microresonator being arranged so as to be at least partially within the core, a first dielectric material having a first refractive index being arranged in a region of the core that is adjacent the microresonator, and a second dielectric material having a second refractive index being arranged on a side of the microresonator opposite the first material.

In accordance with a second aspect of the present invention, there is provided a system for characterising a material, the system comprising:
an optical sensor comprising an optical waveguide, the optical waveguide having first and second ends, the optical sensor further comprising a microresonator, the microresonator comprising an optically active material and being positioned in an optical near field of an end face of the first end of the optical waveguide such that the optically active material is excitable by light, the optical sensor being characterised by having an overlap value greater than or equal to 0.2;
a light source for exciting the optically active material of the microresonator so as to generate WGMs in the microresonator; and
a light collector for collecting an intensity of light that is associated with the WGMs excited in the microresonator.

The system typically is arranged for in-vivo and/or in-vitro biosensing, such as by coating the microresonator with a material that is arranged to interact with a particular biomolecule.

The microresonator may be in contact with the end face of the first end of the optical waveguide, or the microresonator may be spaced from the end face of the first end of the optical waveguide by a distance of 10 μm or less.

It will be appreciated that the end face of the first end of the optical waveguide may have any appropriate orientation. For example, a plane of the end face may be substantially perpendicular with respect to a length of the optical waveguide, or the plane of the end face may be oblique with respect to the length of the optical waveguide. The first end of the optical waveguide may be tapered.

Throughout this specification the term “overlap value” is used for a ratio between a cross-sectional area of light at the first end of the waveguide and an area of the microresonator projected onto the first end of the waveguide.

The overlap value of the optical sensor may be greater than or equal to any one of the group comprising 0.2, 0.4, 0.6, 0.8, 0.9 and 1.0.

The system of the first and second aspects may be arranged for characterising a material that includes, for example, suitable gaseous, solid, and/or liquid materials. In one example the systems are arranged for characterising a material that is a solution or suspension of a material, such as a virus or any other suitable biological material.

The system of the first and second aspects may be arranged for refractive index sensing, environmental sensing, biosensing, temperature sensing, mechanical sensing or any other appropriate sensing of the material.

At least a portion of the system of the first and second aspects may be inserted into a lumen of a catheter, or another appropriate device, so as to facilitate positioning the first end of the optical sensor at a region of interest within a human or other organism.

The first end of the optical sensor may be inserted through the lumen to a delivery end of the catheter, and the second end may be coupled to the light source and the light collector. In this way, the catheter can be used to diagnose and/or monitor disease and/or deliver treatment to a site while the optical sensor is used to sense characteristics of the site to monitor the effectiveness of the treatment.

Alternatively, at least a portion of the system of the first and second aspects may be embedded within a catheter. For example, a catheter may be formed such that the first end of the optical sensor is located and fixed at a position within the catheter that coincides with a delivery end of the catheter, and the second end of the optical sensor is located so as to be couplable to the light source and the light collector.

In accordance with a third aspect of the present invention, there is provided a method of characterising a material, the method comprising the steps of:
providing a system for characterising a material, the system comprising:
   an optical sensor comprising an optical waveguide, the optical waveguide having first and second ends and being characterised by having a numerical aperture greater than or equal to 0.2, the optical sensor further comprising a microresonator, the microresonator comprising an optically active material and being positioned in an optical near field of an end face of the first end of the optical waveguide such that the optically active material is excitable by light;
   a light source for exciting the optically active material of the microresonator so as to generate WGMs in the microresonator; and
   a light collector for collecting an intensity of light; exposing a surface of the microresonator to a material;
directing light from the light source to the microresonator so as to excite the optically active material of the microresonator so as to generate whispering gallery modes (WGMs) in the microresonator;
collecting an intensity of light at the light collector, the intensity of light being associated with the WGMs generated in the microresonator; and
analysing the collected light so as to characterise the material;
wherein the waveguide is used to perform at least one of the steps of directing light to the microresonator and collecting the intensity of light.

In one example, the method is used for in-vivo and/or in-vitro biosensing and the method comprises the step of coating at least a portion of the microresonator with a material that is arranged to interact with a particular biomolecule. The method may be used in endoscopy, fertility monitoring or any other appropriate in-vivo biosensing application.

The step of providing a system for characterizing a material may comprise providing a system wherein the microresonator is in contact with the end face of the first end of the optical waveguide, or wherein the microresonator is spaced from the end face of the first end of the optical waveguide by a distance of 10 μm or less.

Using a waveguide characterised by having a numerical aperture greater than or equal to 0.2 to perform at least one of the steps of directing light to the microresonator and collecting the intensity of light provides the significant advantage of increasing the relative intensity of the collected light compared to conventional methods of characterising a material, such as using a confocal microscope to excite the microresonator and to collect the light.

In one embodiment, the waveguide is used to perform each of the steps of directing light to the microresonator and collecting the intensity of light.

The optically active material is typically a material which absorbs light at a certain wavelength and re-emits light at a higher wavelength, for example an organic dye, a quantum dot, or a rare earth ion. In one specific example, the optically active material is a fluorescent dye, such as Nile Red.

The step of directing light to the microresonator may comprise energising the optically active material to re-emit light that interacts with the microresonator so as to produce a fluorescence pattern that is modulated by the WGMs.

The material that is being characterised may include, for example, suitable gaseous, solid and/or liquid materials. In one example the material is a solution or suspension of a material, such as a virus or any other suitable biological material.

The step of exposing the surface of the microresonator to the material may also comprise functionalising the surface and thereby providing a surface specificity such that predominantly a predetermined biological species, such as a virus, adsorbs at the surface when the surface is exposed to a suitable material. In this case the step of collecting an intensity of light associated with the excited WGMs may comprise detecting a change of a property of the light as a function of adsorbed material and thereby characterising the material.

Alternatively, the step of exposing the surface of the microresonator to the material may also comprise coating the surface with a coating material that is selected so that the material, for example a suitable chemical such as molecule that is capable of selectively cleaving spacer molecules (for example an enzyme), will remove molecules of the coating material from the surface when the surface is exposed to the material. In this case the step of collecting an intensity of light from the interface may comprise detecting a change of a property of the light as a function of removal of coating material and thereby indirectly characterising the material.

The method may comprise the step of operating the microresonator in the lasing regime.

In one embodiment, the optical sensor comprises a plurality of microresonators positioned in the optical near field of the end face of the first end of the waveguide and the method comprises the step of surface functionalising at least two microresonators so as to enable the at least two microresonators to interact with different material particles.

At least some of the microresonators may comprise the same optically active material, such as the same fluorescent dye, such that the at least some of the microresonators emit within the same wavelength range, and the method may comprise the step of exciting the at least some of the microresonators at substantially the same time.

Alternatively, a first group of microresonators may comprise an optically active material that emits within a first frequency range, such as a first fluorescent dye, and a second group of microresonators may comprise an optically active material that emits within a second frequency range and the method may comprise the step of exciting the first group and the second group of microresonators separately.

The waveguide may be an optical fibre having a core diameter that is of the same order as a diameter of the microresonator and comprising a cavity and the method may comprise the steps of:
arranging a first dielectric material having a first refractive index in a region of the cavity; and
arranging the microresonator so as to be at least partially within the core.

The waveguide may be a hollow core MOF.

Such an arrangement, when the microresonator is exposed to a material that comprises or is a constituent of a second dielectric material having a second refractive index, provides the significant advantage of providing an asymmetrical refractive index surrounding the microresonator, thereby resulting in broader resonance features of the microresonator. This may reduce degeneracy of the WGMs.

The method may be used for refractive index sensing, environmental sensing, biosensing, temperature sensing, mechanical sensing or any other appropriate sensing of the material.

### BRIEF DESCRIPTION OF THE DRAWINGS

In order that the present invention may be more clearly ascertained, embodiments of the present invention will now be described, by way of example only, with reference to the accompanying drawings, in which:

FIG. 1 is a schematic diagram of a system for characterising a material in accordance with an embodiment of the present invention;

FIG. 2a is an image of an endface of a waveguide of the system of FIG. 1;

FIG. 2b is an image of the surface of the waveguide shown in FIG. 2b further comprising a microresonator of the system of FIG. 1;

FIG. 3 is a graph showing optical loss measurements of the waveguide of FIG. 1;

FIG. 4 is a schematic diagram of an optical setup used for testing the system of FIG. 1;

FIGS. 5a to 5d are graphs showing results of measurements made using the optical setup of FIG. 4;

FIGS. 6a and 6b are graphs showing results of measurements made using the optical setup of FIG. 4;

FIG. 7 shows a system for characterising a material in accordance with a further embodiment of the present invention;

FIG. 8 is an image of an endface of a waveguide for use in the system shown in FIG. 7;

FIG. 9 is an image of an endface of a waveguide for use in the system shown in FIG. 7;

FIG. 10 illustrates an application in accordance with a specific embodiment of the present invention; and

FIG. 11 is a schematic diagram of a method in accordance with an embodiment of the present invention.

### DETAILED DESCRIPTION OF THE EMBODIMENTS

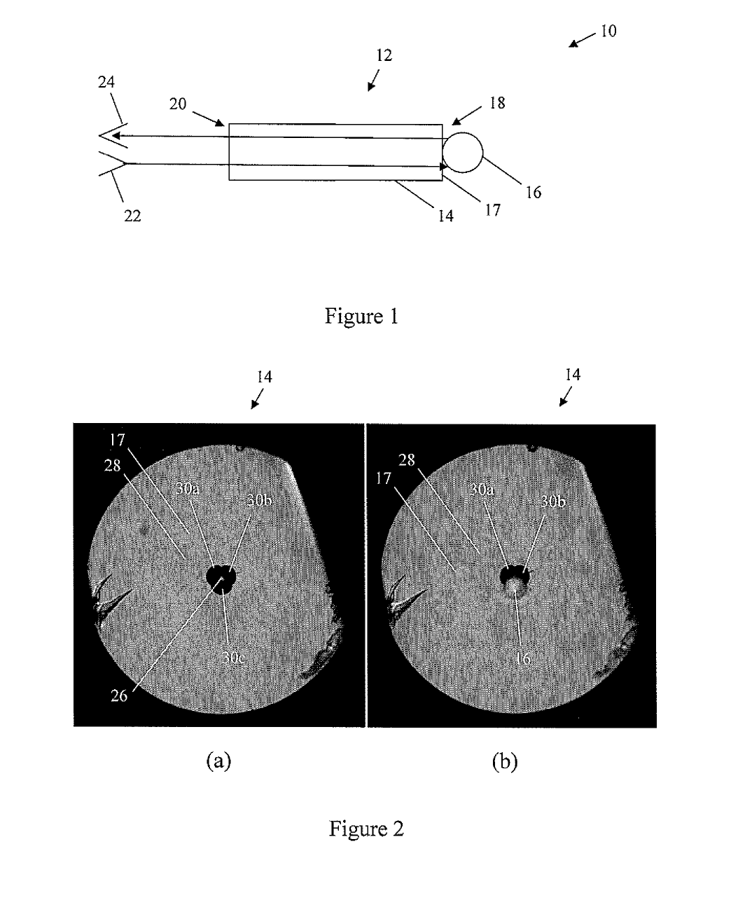

FIG. 1 shows a system 10 that can be used to characterise a material, such as a refractive index of a liquid. The system 10 comprises an optical sensor 12. The optical sensor 12 comprises an optical waveguide 14, in this example a microstructured optical fibre (MOE), and a microresonator 16, in this example a microsphere, comprising an optically active material. The optical waveguide 14 has first and second ends 18, 20 and is characterised by having a numerical aperture greater than or equal to 0.2. The microresonator 16 is positioned in an optical near field of an end face 17 of the first end 18 of the optical waveguide 14 such that the optically active material is excitable by light.

The microresonator 16 may be in contact with the end face 17 of the first end 18 of the optical waveguide 14.

Alternatively, the microresonator 16 may be spaced from the end face 17 of the first end 18 of the optical waveguide 14 by, for example, a distance of 10 μm or less. For example, the end face 17 may be coated with an optically transmissive material, and the microresonator 16 may be in contact the coating rather than being in direct contact with the end face 17.

Further, in the examples that follow, a plane of the end face 17 is substantially perpendicular with respect to a length of the optical waveguide 14, however it will be appreciated that the plane of the end face 17 may be oblique with respect to the length of the optical waveguide 14. Further, in the examples that follow the first end 18 is not tapered, although it will be appreciated that the first end 18 of the optical waveguide 14 may be tapered.

The system 10 also comprises a light source 22 for exciting whispering gallery modes (WGMs) in the microresonator 16 and a light collector 24 for collecting an intensity of light that is associated with the WGMs excited in the microresonator 16.

The system 10 may be arranged such that the light used to excite WGMs in the microresonator 16 is directed to the microresonator 16 via the optical waveguide 14, the system 10 also being arranged such that the intensity of light associated with the WGMs excited in the microresonator 16 is directed to the light collector 24 via the optical waveguide 14. However, it will be appreciated that only one of the light directed towards the microresonator 16 or the light directed to the light collector need be directed via the optical waveguide 14.

The system 10 provides the significant advantage of providing an optical sensor 12 that can function as, for example, a dip sensor, wherein the optical waveguide 14 is used for both directing light to the microresonator 16 so as to excite WGMs in the microresonator 16 and for collecting an intensity of light that comprises at least a portion of the excited WGMs.

This facilitates use of the system 10 in biosensing applications, such as in-vivo sensing and, advantageously, the system 10 can be incorporated into devices such as catheters so as to facilitate positioning the first end 18 (that is, the sensing end) at a region of interest within a human or other organism. In one particular example, the system 10 is embedded into a catheter so as to provide a device that could, for example, deliver a treatment to a particular site, while sensing the characteristics of the site to monitor the effectiveness of the treatment.

Further, having an optical waveguide 14 characterised by having a numerical aperture greater than or equal to 0.2 provides the significant advantage of increasing the excitation and collection efficiency of a WGM signal generated by the microresonator 16 compared to a typical sensor such as a microresonator embedded into a microfluidic flow cell.

The optical sensor 12 is also characterised by having an overlap value greater than or equal to 0.2, the overlap value being defined as a ratio between an area of light exiting the first end 18 of the waveguide 14 and an area of the microresonator 16 projected onto the first end 18.

The overlap value of the optical sensor may be greater than or equal to any one of the group comprising 0.2, 0.4, 0.6, 0.8, 0.9 and 1.0.

The overlap between the light exiting the waveguide 14 and the microresonator 16 positioned at the first end 18 of the microresonator 16 can be approximated by:

$$𝒪 = \frac{{A}_{eff} {\vert}_{{A}_{res}}}{max ( {A}_{res} , {A}_{eff} )} \qquad Equation 2$$

where Aᵣₑₛ is the projected area of the microresonator 16 on the plane of the endface 17 of the first end 18 of the waveguide 14 and where:

$${A}_{eff} {\vert}_{{A}_{res}} = \frac{{( {\int }_{{A}_{res}} {\vert E ( r ) \vert}^{2} d {r}^{2} )}^{2}}{{\int }_{{A}_{res}} {\vert E ( r ) \vert}^{4} d {r}^{2}} \qquad Equation 3$$

is the effective area of the guided light residing within the resonator region Aᵣₑₛ. This expression (Equation 2) for [symbol] calculates the fraction of the effective area of the guided light residing within an area of the microresonator 16 (projected onto the endface 17 of the first end 18), normalised to the area of either the light or the resonator area (whichever is larger). [symbol] →1 for an input beam positioned at the centre of, and the same effective area as, the microresonator 16. For an input beam smaller or larger than the area of the microresonator 16 or a microresonator 16 offset from the beam, [symbol] decreases in value ( [symbol] →0).

Numerical aperture values of interest for the system 10 are generally greater than or equal to 0.2. Particular waveguides 14 used in experiments with the system 10 have a numerical aperture of approximately 1.25 to 1.75. Numerical aperture values could be higher, for example in the order of 3.0.

The microresonator 16 comprises an optically active material. In the examples that follow, the optically active material is Nile red, a fluorescent dye material. It will be appreciated, however, that the optically active material may be any appropriate optically active material such as a material which absorbs light at a certain wavelength and re-emits light at a higher wavelength, for example an organic dye, a quantum dot, or a rare earth ion.

In this particular example, the microresonator 16 is a polystyrene microsphere having a diameter of 10 μm (ΔØ=0.8 μm, n=1.59) and was doped with a fluorescent laser dye (Nile red) using a liquid two-phase system. The procedure for forming such polystyrene microspheres will now be described.

The fluorescent dye was first dissolved into xylene until the solubility limit was reached. The resulting solution was poured on top of an aqueous suspension of microspheres and agitated with a magnetic stirrer until the xylene completely evaporated. As the xylene and deionised water are immiscible, as the xylene evaporates, the fluorescent dye is transferred into the microspheres that come into contact with the dye solution.

After the doping procedure, the microsphere solution was annealed within a hermetically sealed container above the boiling temperature of the xylene for 2 hours in order to remove traces of solvent from the microspheres. The microspheres were then washed by centrifugation, the supernatant removed and the lost volume of the deionised water replaced.

In this example, and as shown in FIGS. 2a and 2b, the optical waveguide 14 is a MOF fabricated from a lead-silicate glass (n=1.62 @ 546.1 nm). The optical waveguide has a core 26 having a diameter of Ø_{core}˜1.5 μm, providing strong light confinement, surrounded by a cladding region 28 and three relatively large holes 30a, 30b, 30c having a diameter (Øₕₒₗₑ˜5 μm) on which the microresonator 16 can be located.

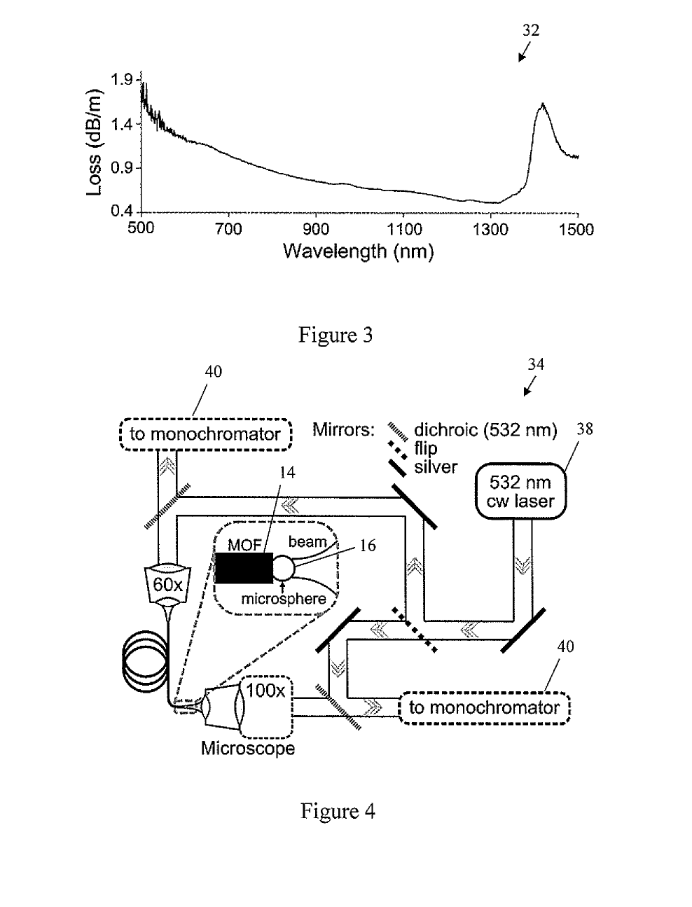

The waveguide 14 also has a relatively high numerical aperture, which increases the fluorescence capture efficiency of the system 10. A typical optical loss spectrum of this fibre is shown in graph 32 of FIG. 3, showing that, although the maximum transmission band is within the near infra-red region (near 1.3 μm), the losses in the visible are still relatively low (1.4 dB/m @ 532 nm).

The microresonator 16 can be positioned onto the end face 17 of the first end 18 of the optical waveguide 14. In this example the microresonator 16 was positioned onto the end face 17 of the first end 18 by using a translation stage. In particular a microscope glass cover slip, aligned using the translation stage, was smeared with a drop of the microsphere solution. A microsphere was selected from the many deposited onto the slide by qualitatively analysing its emission spectrum via excitation and collection using a confocal microscope. Once a suitable microsphere was found, it was put into contact with a cleaved tip of a 20 cm long waveguide 14 which was aligned using a microscope stage. In this example, and as shown in FIG. 2b, the microresonator 16 coupled with the waveguide 14 at or near the hole 30c.

To assess the increase of excitation and collection efficiency when the microresonator 16 is positioned at the end face 17 of the first end 18 of the waveguide 14, an optical setup 34, shown in FIG. 4, allowing both the excitation and the collection through either the waveguide 14 or a confocal microscope 36 was arranged.

The microresonator 16, a microsphere containing a fluorescent dye (Nile red), was first positioned onto the end face 17 of the first end 18 of the waveguide 14, a MOF. The excitation was performed with a CW 532 nm laser 38 while the fluorescence spectra was analysed using a Jobin-Yvon/Horiba monochromator 40 comprising a CCD camera.

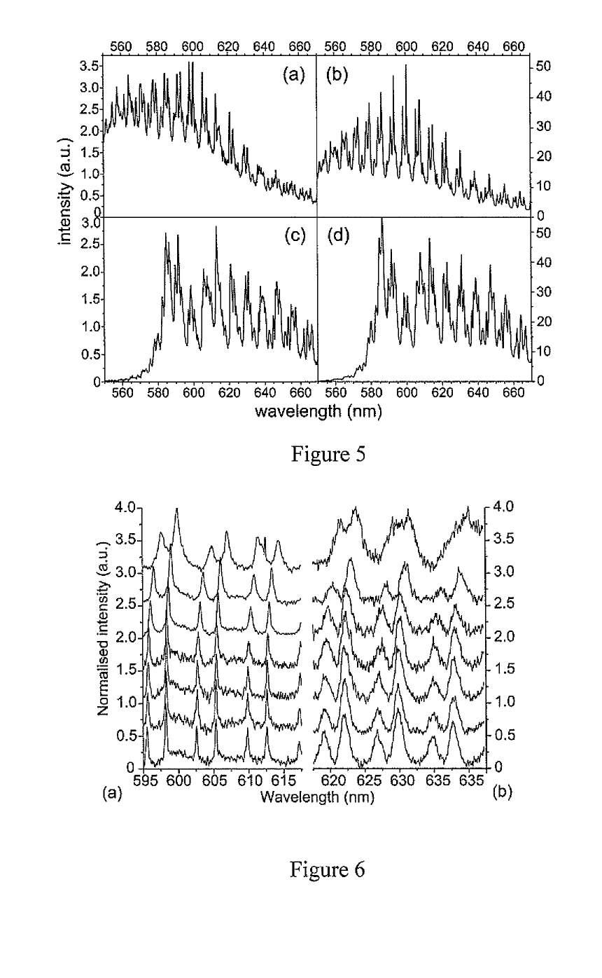

In a first test, the results of which are shown in FIG. 5a, both the excitation and the signal collection were performed using the confocal microscope 36, which yielded a measured excitation power of 77 μW at an objective output of the microscope 36.

In a second test, results of which are shown in FIG. 5b, the configuration was similar to the first test except that the excitation was performed through the waveguide 14, with an excitation power of 3 μW measured at the first end 18 of the waveguide 14, and a fluorescence signal was again collected by the objective of the microscope 36. The lower excitation power measured at the first end 18 of the waveguide 14 compared to that measured at the objective of the microscope 36 is mainly due to the high losses induced by the low coupling efficiency of the laser 38 into the waveguide 14 and the losses of the waveguide 14 itself at 532 nm (˜1.4 dB/m).

Nevertheless, in both cases WGMs can still be observed. More importantly, as shown in FIGS. 5a and 5b, the relative intensity of the fluorescence signal is significantly higher when the waveguide 14 is used for excitation, rather than the objective of the microscope 36. Indeed, a ≈9.2 fold increase of the integrated spectra is observed.

The results shown in FIGS. 5c and 5d were obtained using the same microresonator 16, but with the fluorescence signal collected by the waveguide 14 and with excitation via the objective of the microscope 36 or the waveguide 14, respectively. The fluorescence intensity is again much higher when the microresonator 16 is excited using the waveguide 14, but now with a ≈19 fold increase of the integrated signal. This demonstrates that the use of a high numerical aperture waveguide 14 increases both the efficiency of excitation and collection of the WGMs.

To assess the sensitivity of the microresonator 16 positioned at the end face 17 of the first end 18 of the waveguide 14, and its potential application for refractive index dip sensing, the WGM spectra were also recorded when the first end 18 of the waveguide 14 was dipped into water/glycerol solutions with increasing glycerol concentrations (see FIG. 6b). These spectra were compared to another microresonator 16 that was prepared from a same batch and that was attached to a glass slide within a microfluidic flow cell (see FIG. 6a).

In both cases, when the liquid surrounds the microresonator 16, the higher order modes are quenched due to the large decrease in refractive index contrast compared to the dry/air case, resulting in spectra with the typical periodic repetition of first order TE and TM modes. Both microresonators 16 exhibited similar sensitivities, 56.93 nm/RIU and 45.49 nm/RIU for the waveguide 14 and flow-cell versions respectively (with a linear regression coefficient over 0.99 in both cases).

The difference of sensitivity may be due to the slight difference in diameter of the two microresonators 16 (which was confirmed by analysing the mode spacing), rather than the excitation/collection scheme. It was observed that the Q factor (Q−λ/Δλ) of the microresonator 16 deposited onto the waveguide 14 is significantly lower (Q˜500) compared to the microresonator 16 embedded within the microfluidic flow cell (Q˜1000).

Furthermore, the Q factor of the microresonator 16 on the waveguide 14 decreases rapidly as the index increases around the microresonator 16, down to Q˜300 for the 25% glycerol solution. As the glycerol concentration increases, the solution becomes more viscous and it is possible that the diffusion of the glycerol solution around the microresonator 16 is affected by the waveguide 14 itself since the microresonator 16 sits partially across one of the holes 30c, resulting in an inhomogeneous refractive index distribution on the microresonator 16 surface. Such a distribution will result in a loss of degeneracy of the WGMs and consequently a broadening of the observed modes, as observed.

For a microresonator 16 comprising an optically active material, the microresonator 16 can be operated in the lasing regime.

Having an optical sensor comprising a microresonator 16 arranged to operate in the lasing regime provides the significant advantage of increasing the Q factor of the microresonator 16 and therefore a sensitivity at which the microresonator 16 reacts to changes in its environment, and may induce an electromagnetic field around the microresonator 16 which may attract material particles to the surface of the microresonator 16, thereby resulting in a faster binding kinetic between the surface of the microresonator 16 and the material particles. Further, the lasing threshold of the microresonator 16 may be lowered due to its positioning at or near the end face 17 of the first end 18 of the waveguide 16 and the resulting increase of an excitation efficiency of the microresonator 16.

In an alternative embodiment to the examples discussed above, the waveguide 14 may be a multi-core optical fibre and the system 10 may be arranged such that a first core is used in the excitation of WGMs in the microresonator 16 and a further core is used in collecting an intensity of light that is associated with the WGMs excited in the microresonator 16.

In addition to the examples discussed above, the microresonator 16 may be coupled to a resonator, such as a further microresonator.

In addition to the examples discussed above wherein the optical sensor 12 comprises a single microresonator 16, it will be appreciated that the optical sensor 12 may comprise a plurality of microresonators 16 coupled at or near the end face 17 of the first end 18 of the waveguide 14. At least two of these microresonators 16 can be arranged so as to interact with different material particles.

In one example, each microresonator 16 is surface functionalised so as to enable each microresonator 16 to interact with a different material particle. Each microresonator 16 may comprise the same optically active material, such as the same fluorescent dye, such that each microresonator 16 emits within the same wavelength range. In an alternative embodiment, each microresonator 16 comprises an optically active material that emits within a different frequency range, such as a different fluorescent dye, thereby allowing each microresonator 16 to be excited separately.

In the above examples, the waveguide 14 comprises a MOF having a solid core and a wagon wheel, or small core microstructured optical fibre architecture. It will also be appreciated that the waveguide 14 may be a MOF comprising a hollow core. An embodiment wherein the waveguide is a MOF comprising a hollow core will now be described.

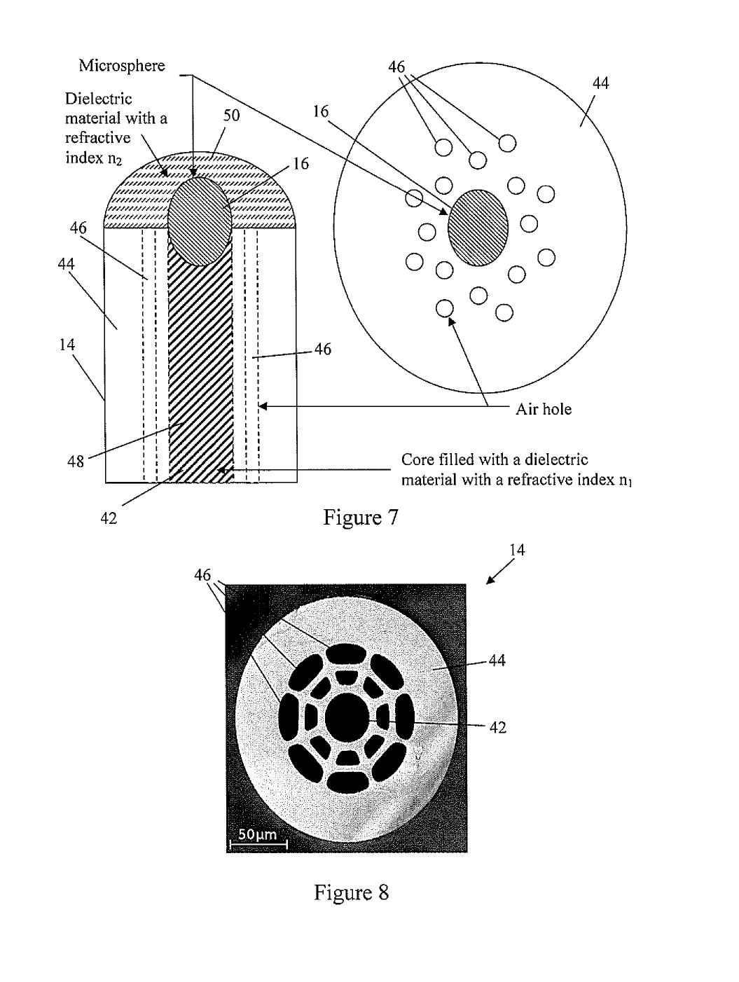

In one embodiment, shown in FIG. 7, the waveguide 14 is a hollow core fibre comprising a hollow core 42 having a core diameter that is of the same order as a diameter of the microresonator 16, the microresonator 16 being arranged so as to be at least partially within the core 42. The core 42 is surrounded by a cladding 44, and a plurality of air holes 46 extending through the length of the fibre. A first dielectric material 48 having a first refractive index is arranged in a region of the core 42 that is adjacent the microresonator 16, and a second dielectric material 50 having a second refractive index is arranged on a side of the microresonator 16 opposite the first dielectric material 48.

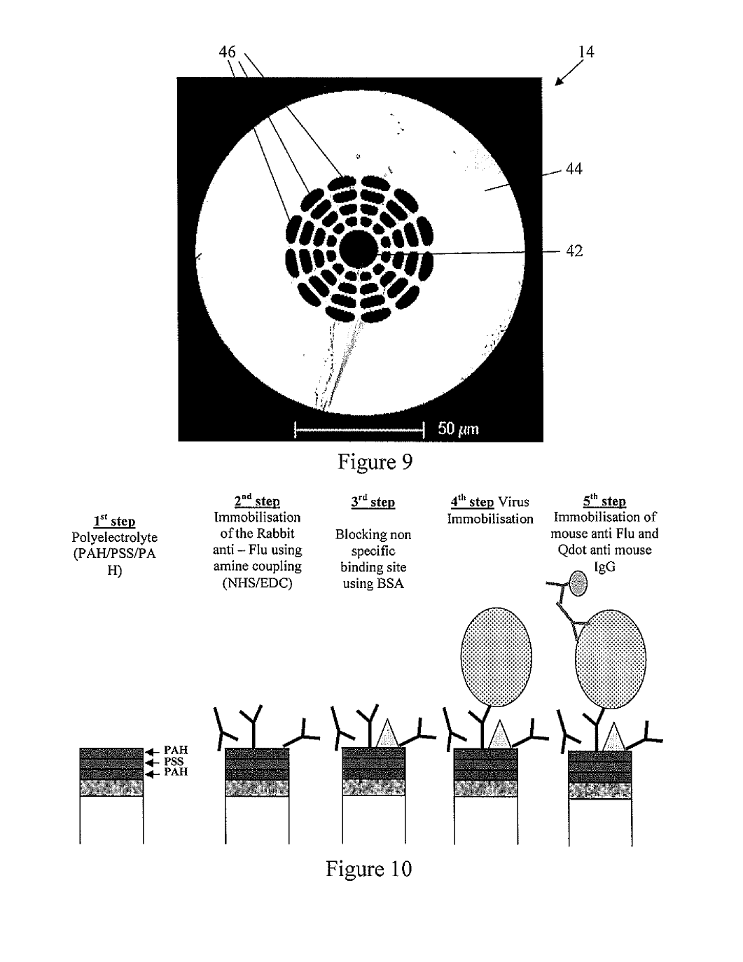

Further hollow core waveguides 14 that would be appropriate for the arrangement shown in FIG. 7 are shown in FIGS. 8 and 9. FIG. 8 shows a hollow core waveguide 14 having a core 42 surrounded by cladding 44 and a plurality of air holes 46 arranged in two rings around the core 42. FIG. 9 shows a hollow core waveguide 14 having a core 42 surrounded by cladding 44 and a plurality of air holes 46 arranged in four rings around the core 42.

The system 10 may be arranged for characterising a material that includes, for example, suitable gaseous, solid, and/or liquid materials. The system 10 may be arranged for characterising a material that is a solution or suspension of a material, such as a virus or any other suitable biological material.

The system 10 may be arranged for refractive index sensing, environmental sensing, biosensing, temperature sensing, mechanical sensing or any other appropriate sensing of the material.

As mentioned above, the system 10 can be used for biosensing, and is appropriate for both in-vivo and in-vitro biosensing applications. In-vivo and in-vitro biosensing applications can be facilitated by coating the microresonator with a material that is arranged to interact with a particular biomolecule

In one example, at least a portion of the system 10 is inserted into a lumen of a catheter, or other appropriate device, so as to facilitate positioning the first end 18 of the system 10 at a region of interest within a human or other organism. For example, the first end 18, comprising the microresonator 16, can be inserted through the lumen to a delivery end of the catheter and the second end coupled to the light source 22 and the light collector 24. In this way, the catheter can be used to deliver treatment to a site while the system 10 is used to sense characteristics of the site to monitor the effectiveness of the treatment. The treatment can be delivered via the lumen if insertion of the system 10 into the lumen provides sufficient space, or via a further lumen, for example if the catheter is a two lumen catheter.

It is envisaged that at least a portion of the system 10 can be embedded within a catheter. For example, a catheter may be formed such that the first end 18 of the system 10 is located and fixed at a position within the catheter that coincides with a delivery end of the catheter. The second end 20 is located so as to be couplable to the light source 22 and the light collector 24.

In this way, a single device that is capable of both delivering treatment to a site within a human or other organism, and sensing characteristics of the site to measure an effectiveness of the delivered treatment is provided.

It will further be appreciated that a system 10/catheter device can be used in endoscopy, fertility monitoring or any other appropriate biosensing application.

Surface functionalisition of the microresonator 16 will now be described with reference to FIG. 10. Initially a polyelectrolyte coating, comprising a PAH (PolyAllylamine Hydrochloride) layer followed by a PSS layer and then another PAH layer was applied to the surface of the microresonator (1^{st} step) using the layer by layer deposition technique, providing amine functional groups on the coating surface, then an Rabbit anti-flu antibody was immobilised onto the surface using amine coupling reagents EDC/NHS (EDC: 1-Ethyl-3-[3-dimethylaminopropyl]carbodiimide hydrochloride; NHS: N-hydroxysuccinimide) (2^{nd} step). Non-specific binding states were blocked using BSA (Bovine Serum Albumin) (5%) (3^{rd} step), a swine flu virus was then immobilized (4^{th} step), specifically interacting with the rabbit anti-flu antibody and subsequently a mouse anti-flu antibody followed by a Qdot labelled anti mouse antibody were immobilized (5^{th} step) in order to finalise a sandwich assay and confirm the presence of the swine flu virus onto the surface. The sensor was rinsed between each step using PBS buffer at pH 7.4.

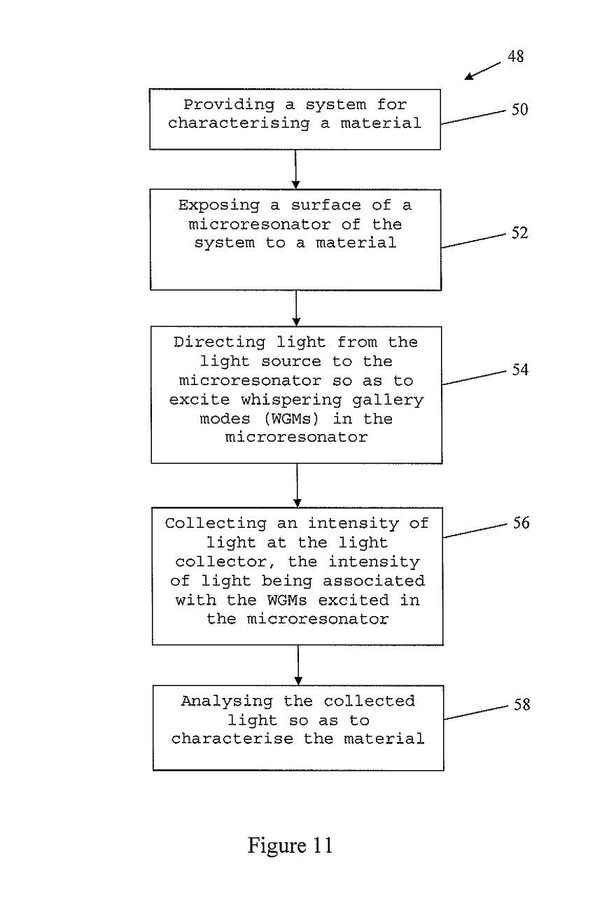

A method 48 of characterising a material using the system 10 will now be described with reference to FIG. 11. The method comprises a first step 50 of providing the system 10 for characterising a material, a second step 52 of exposing a surface of the microresonator 16 to a material, a third step 54 of directing light from the light source 22 to the microresonator 16 so as to excite whispering gallery modes (WGMs) in the microresonator 16, a fourth step 56 of collecting an intensity of light at the light collector 24, the intensity of light being associated with the WGMs excited in the microresonator 16 and a fifth step 58 of analysing the collected light so as to characterise the material. The waveguide 14 of the system 10 is used to perform at least one of the third step 54 step of directing light to the microresonator 16 or the fourth step 56 of collecting the intensity of light.

Using a waveguide 14 characterised by having a numerical aperture greater than or equal to 0.2 to perform at least one of the steps 54, 56 of directing light to the microresonator and collecting the intensity of light provides the significant advantage of increasing the relative intensity of the collected light compared to conventional methods of characterising a material, such as using a confocal microscope to excite the microresonator and to collect the light.

In one embodiment, the waveguide 14 is used to perform each of the steps 54, 56 of directing light to the microresonator and collecting the intensity of light.

In one example, the microresonator 16 comprises an optically active material such as a fluorescent material or quantum dots and the third step 54 of directing light to the microresonator comprises energising the optically active material to re-emit light that interacts with the microresonator 16 so as to produce a fluorescence pattern that is modulated by the WGMs.

The material that is being characterised may include, for example, suitable gaseous, solid and/or liquid materials. In one example the dielectric material is a solution or suspension of a material, such as virus or any other suitable biological material.

The second step 52 of exposing the surface of microresonator 16 to the material may also comprise functionalising the surface and thereby providing a surface specificity such that predominantly a predetermined biological species, such as a virus, adsorbs at the surface when the surface is exposed to a suitable material. In this case the fourth step 56 of collecting an intensity of light associated with the excited WGMs may comprise detecting a change of a property of the light as a function of adsorbed material and thereby characterising the material.

Alternatively, the second step 52 of exposing the surface of the microresonator 16 to the material may also comprise coating the surface with a coating material that is selected so that the material, for example a suitable chemical such as molecule that is capable of selectively cleaving spacer molecules (for example an enzyme), will remove molecules of the coating material from the surface when the surface is exposed to the material. In this case the fourth step 56 of collecting an intensity of light from the interface may comprise detecting a change of a property of the light as a function of removal of coating material and thereby indirectly characterising the material.

In one embodiment, the method 48 comprises the step of operating the microresonator 16 in the lasing regime.

Operating the microresonator 16 in the lasing regime provides the significant advantage of increasing a sensitivity at which the microresonator 16 reacts to changes in its environment, and may induce an electromagnetic field around the microresonator 16 which may attract material particles to the surface of the microresonator 16, thereby resulting in a faster binding kinetic between the surface of the microresonator 16 and the material particles. Further, a lasing threshold of the microresonator 16 may be lowered due to its positioning at or near the end face 17 of the first end 18 of the waveguide 14 and the resulting increase in its excitation efficiency.

In one embodiment, the optical sensor 12 comprises a plurality of microresonators 16 optically coupled at or near the end face 17 of the first end 18 of the waveguide 14 and the method 48 comprises the step of surface functionalising each microresonator 16 so as to enable each microresonator 16 to interact with a different material particle.

Each of the plurality of microresonators 16 may comprise the same optically active material, such as the same fluorescent dye, such that each microresonator 16 emits within the same wavelength range, and the method 48 may comprise exciting at least a portion of the microresonators 16 at substantially the same time.

Alternatively, each of the plurality of microresonators 16 may comprise an optically active material that emits within a different frequency range, such as a different fluorescent dye, and the method 48 may comprise exciting one or more of the microresonators 16 separately.

The waveguide 14 may be a hollow core fibre (see FIG. 7) having a core 42 having a diameter that is of the same order as a diameter of the microresonator 14 and the method 48 may comprise the steps of:
arranging a first dielectric material 48 having a first refractive index in a region of the core that is near or adjacent a first end of the microresonator 16; and
arranging the microresonator 16 so as to be at least partially within the core 42.

Such an arrangement, when the microresonator 16 is exposed to a material that comprises or is a constituent of a second dielectric material 50 having a second refractive index, provides the significant advantage of providing an asymmetrical refractive index surrounding the microresonator 16, thereby resulting in broader resonance features of the microresonator 16. This may reduce degeneracy of the WGMs.

The method 48 may be used for refractive index sensing, environmental sensing, biosensing, temperature sensing, mechanical sensing or any other appropriate sensing of the material.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

Although the invention has been described with reference to particular examples, it will be appreciated by those skilled in the art that the invention may be embodied in many other forms.

1.-54. (canceled)

55. A system for characterising a material, the system comprising:
an optical sensor comprising an optical waveguide, the optical waveguide having first and second ends and being characterised by having a numerical aperture greater than or equal to 0.2, the optical sensor further comprising a microresonator, the microresonator comprising an optically active material and being positioned in an optical near field of an end face of the first end of the optical waveguide such that the optically active material is excitable by light;
a light source for exciting the optically active material of the microresonator so as to generate whispering gallery modes (WGMs) in the microresonator; and
a light collector for collecting an intensity of light that is associated with the WGMs excited in the microresonator.

56. The system of claim 55, wherein the optically active material is a fluorescent dye.

57. The system of claim 55, wherein the optically active material is a rare earth doped material.

58. The system of claim 55, wherein the optical waveguide is an optical fibre.

59. The system of claim 55, wherein the waveguide is a microstructured optical fibre (MOF).

60. The system of claim 55, wherein the waveguide is a multi-core optical fibre and the system is arranged such that a first core is used in the excitation of WGMs in the microresonator and a further core is used in collecting an intensity of light that is associated with the WGMs excited in the microresonator.

61. The system of claim 55, wherein the microresonator is a microsphere.

62. The system of claim 61, wherein the microresonator has a diameter in the range of any one of the ranges comprising 1 μm-50 μm, 5 μm-15 μm.

63. The system of claim 55, wherein the microresonator is arranged so as to be operable in the lasing regime.

64. The system of claim 55, wherein the sensor comprises a plurality of microresonators positioned in an optical near field of an end face of the first end of the waveguide, at least two microresonators being arranged so as to interact with different material particles.

65. The system of claim 64, wherein at least some microresonators are surface functionalised so as to enable the at least some microresonators to interact with the same and/or different material particles.

66. The system of claim 64, wherein a first group of microresonators comprise an optically active material that emits within a first frequency range, and a second group of microresonators comprise an optically active material that emits within a second frequency range such that each of the first and second groups of microresonators may be excited separately.

67. The system of claim 55, wherein the waveguide is a hollow core fibre having a core diameter that is of the same order as a diameter of the microresonator, the microresonator being arranged so as to be at least partially within the core, a first dielectric material having a first refractive index being arranged in a region of the core that is adjacent the microresonator, and a second dielectric material having a second refractive index being arranged on a side of the microresonator opposite the first material.

68. The system of claim 55, wherein the system is arranged for refractive index sensing, environmental sensing, biosensing, temperature sensing, mechanical sensing or any other appropriate sensing of the material.

69. The system of claim 55 wherein the system is arranged for in-vivo and/or in-vitro biosensing.

70. The system of claim 55, wherein at least a portion of the system is embedded within a catheter.

71. A method of characterising a material, the method comprising the steps of:
providing a system for characterising a material, the system comprising:
an optical sensor comprising an optical waveguide, the optical waveguide having first and second ends and being characterised by having a numerical aperture greater than or equal to 0.2, the optical sensor further comprising a microresonator, the microresonator comprising an optically active material and being positioned in an optical near field of an end face of the first end of the optical waveguide such that the optically active material is excitable by light;
a light source for exciting the optically active material of the microresonator so as to generate WGMs in the microresonator; and
a light collector for collecting an intensity of light;
exposing a surface of the microresonator to a material;
directing light from the light source to the microresonator so as to excite the optically active material of the microresonator so as to generate whispering gallery modes (WGMs) in the microresonator;
collecting an intensity of light at the light collector, the intensity of light being associated with the WGMs generated in the microresonator; and
analysing the collected light so as to characterise the material;
wherein the waveguide is used to perform at least one of the steps of directing light to the microresonator and collecting the intensity of light.
